(19) United States
(12) Patent Application Publication
(10) Pub. No.: US 2005/0181018 A1
(43) Pub. Date: Aug. 18, 2005

(54) Ocular drug delivery

(76) Inventor: Gholam A. Peyman, New Orleans, LA (US)

Correspondence Address: WOOD, HERRON & EVANS, LLP, 2700 CAREW TOWER, 441 VINE STREET, CINCINNATI, OH, 45202, US

(21) Appl. No.: 11/105,756

(22) Filed: Apr. 14, 2005

Related U.S. Application Data
(63) Continuation-in-part of application No. 10/752,124, filed on Jan. 6, 2004.
(63) Continuation-in-part of application No. 10/667,161, filed on Sep. 19, 2003.

(51) Int. Cl. A61F 2/00

(52) U.S. Cl. 424/427; 604/294

(57) ABSTRACT

An ocular device that contains a drug, the device affixed to the sclera for release of drug into the sclera through at least one opening. Control of drug release may be mechanical and/or electrical, and may be at a point of use or from a remote location. In one embodiment, an electrode is affixed to the sclera opposite the device, and initiation of current releases the drug from the device via iontophoresis. The lumen or reservoir contents may be visualized, and/or an indicator may indicate content information such as volume. In one embodiment, a multicompartment device may controllably release the same drug at a different rate or different drugs at the same or different rates.

This application is a Continuation-In-Part of U.S. patent application Ser. No. 10/752,124 filed Jan. 6, 2004, pending; which is a Continuation-In-Part of U.S. patent application Ser. No. 10/667,161 filed Sep. 19, 2003, pending, each of which is expressly incorporated by reference herein in its entirety.

### FIELD OF THE INVENTION

The invention is directed to scleral depot delivery of an agent such as a drug to the posterior segment of the eye.

### BACKGROUND

The eye is naturally bathed internally and externally by ocular fluids. The external portion of the eye is lubricated by lacrimal fluids (tears). The internal portion of the eye has two fluid-containing chambers: the anterior chamber contains the aqueous humor or aqueous, and the posterior chamber contain the vitreous humor or vitreous. The wall of the eyeball has three layers: (1) the outer protective, tough, and fibrous corneoscleral coat; (2) the middle vascular uvea; and (3) the inner photosensitive retina. The outermost corneoscleral coat is divided into the sclera, which is the larger opaque posterior segment, and the cornea, which is the smaller transparent anterior segment.

### SUMMARY OF THE INVENTION

One embodiment is a method for ocular drug delivery by providing a delivery device having at least one opening for release of an agent, such as a drug, contained within a lumen of the device, and fixing the device to the sclera such that the agent is released to the sclera through the opening. The device may be pre-set to release agent, or release may be regulated at a point of use or a remote location. An electrically-disruptable material may associated with the opening, and an electrical stimulus can be used to disrupt the material to release the agent. In another embodiment, an electrode can be fixed to the sclera substantially opposite the device, with a current generated to release the agent by iontophoresis. The device may have one or more compartments, each compartment may contain a different agent, have its contents released at a different rate, etc. The device may be positioned so that it can be monitored and refilled while in use.

One embodiment is an ocular drug delivery device that has at least one lumen for containing an agent, at least one controllable opening, a subtantially linear body for affixing to the sclera in approximation to a conjunctiva or for locating within a tunnel created in the sclera, with the device capable of controllably releasing agent contained in the lumen from the opening. The device may have tapered ends that assist in located or securing it in the eye. The device may include implements that allow the release of agent to be controlled.

These and other embodiments of the invention will be apparent in light of the following figures and detailed description.

### BRIEF DESCRIPTION OF THE FIGURES

FIG. 1 is a schematic view of the eye with the device in place.

FIG. 2 is a side view of an embodiment of the device in place within the eye.

FIG. 3 is a cross-sectional view of the device of FIG. 2 generally taken along line 3-3.

FIG. 4 is a partially torn away bottom view of another embodiment of the device in place within the eye.

FIG. 5 is a cross-sectional view of the device of FIG. 4 generally taken along line 5-5.

FIG. 6 is a cross-sectional view of another embodiment of the device showing a self-sealing wall penetrated by a needle for introducing agent into the device.

FIG. 7 is a cross-sectional view of another embodiment of the device showing an injection port receiving a needle for introducing agent into the device.

FIG. 8 is a side view of another embodiment of the device showing openings that vary in size.

FIG. 9 is a side view of another embodiment of the device showing multiple compartments containing different agents.

FIG. 10 is a side view of another embodiment of the device having a curved shape and placed within the eye.

### DETAILED DESCRIPTION

A device and method in which the sciera is used to deliver an agent to the posterior segment of the eye is disclosed. In one embodiment, the agent may be an ocular solution containing one or more macrolide antibiotics and/or mycophenolic acid. The ocular solution may be any physiologically compatible ocular solution. It may used externally (e.g. topical administration such as on the surface of the conjunctiva) or, using the inventive method and device, internally (e.g. invasive administration).

Ocular solutions are frequently administered to a patient following ocular surgery; macrolide antibiotics in these solutions desirably provide anti-inflammatory effects that aid in post-surgical recovery. In addition, macrolide antibiotics provide these anti-inflammatory effects without an increase in intraocular pressure that often accompanies administration of steroids to post-surgical patients to control inflammation.

Macrolide antibiotics also reduce cell proliferation and cell migration. This may promote the healing process, and may also provide an anti-angiogenesis effect to retard the proliferation and/or growth of new vessels. As one example, controlling the growth of new blood vessels is a way to control proliferation of tumor cells; macrolide antibiotics in an ocular solution may be helpful in controlling ocular neoplasms or tumors. As another example, the solutions may be used in patients having diseases characterized by abnormal angiogenesis, such as certain types of cancers, diabetic retinopathy, and sickle cell retinopathy, in which an anti-angiogenesis effect is desirable. Macrolide antibiotics also provide antimicrobial and antifungal properties to ocular solutions.

Macrolide antibiotics and/or mycophenolic acid may be used to enhance therapy in ocular diseases. Such enhancement is generally defined as treatment of these diseases. Treatment is not limited to total elimination of disease, but is broadly defined to include any enhancement or improvement toward the result of diminishing or alleviating the disease symptoms, onset, course, duration, severity, etc. Macrolide antibiotics and/or mycophenolic acid may be combined with other agents, such as chemotherapeutic agents for treatment of ocular malignancies, and cyclooxygenase inhibitors for reducing inflammation. Both acute and chronic ocular diseases are treated by the inventive method and composition, and include retinitis pigmentosa, diabetic retinopathy, age related macular degeneration, scleritis, uveitis, and vasculitis. Ocular cancers such as retinoblastoma, choroidal melanoma, pre-malignant and malignant conjunctival melanoma are also treated by the invention.

It will be appreciated that the composition need not be in the physical form of a true solution, but instead may be a suspension, an emulsion, a gel, etc. It may also encompass the macrolide antibiotic and/or mycophenolic acid in the form of polymeric compositions, microspheres, microvesicles, microcapsules, and/or liposomes or in nanotechnology formulations. In addition, ocular solutions for topical application may take the form of any of the above, as well as an ointment, a cream, a lotion, etc. Thus, the term solution is used for convenience but encompasses other physical states. It will also be appreciated that the macrolide antibiotics may be included in the formulation for preparing an ocular solution, or may be added in dry form or in concentrated form to an already prepared ocular solution.

The ocular solution may be one that is used as an ocular irrigating solution and/or as a volume replacement solution during ocular surgery. It is thus a substitute for an ocular fluid, such as the vitreous, and/or a substitute for a commercially available irrigating solution that may be used during ocular surgery. It may also be one that is used topically, and thus encompasses eye drops, eye wash solutions, and contact lens solutions. It may be used in over the counter (OTC) ocular solutions for topical application, for example, in ocular solutions such as artificial tears or lubricants. One commercially available ophthalmic lubricant is Viva-Drops®, available from Vision Pharmaceuticals, Inc. (Mitchell SD). The invention includes but is not limited to this particular embodiment.

In one embodiment, an ocular solution contains at least one macrolide antibiotic and/or mycophenolic acid and is used for intraocular administration. Intraocular administration indicates an invasive route of administration, compared to a topical route of administration. An invasive route of administration therefore encompasses various degrees of invasiveness, including minimally invasive routes. In this embodiment, the ocular solution containing the macrolide antibiotic(s) may be an irrigating solution, a volume replacement solution, and/or a wash solution.

In another embodiment, an ocular solution containing a macrolide antibiotic and/or mycophenolic acid is administered topically, for example, on the conjunctiva or the mucosal surface of the lid, to treat diseases in other areas of the eye, such as the choroid, retina, and uvea. Administration of such compounds was previously restricted to systemic or invasive routes, because it was thought that the higher concentrations of these compounds in internal ocular structures required for efficacy could not be achieved by topical administration. However, an efficacious therapeutic concentration of a topically-administered macrolide antibiotic and/or mycophenolic acid in an ocular structure may be achieved by topically administering a supratherapeutic concentration for a duration such that a therapeutic concentration is attained in the diseased structure.

While not bound by any theory, one reason this therapeutic concentration may be achieved with topical administration is that the structural affinity of the antibiotic and/or mycophenolic acid for lipids results in their accumulation in lipophilic regions of the choroid, retina, etc. Unexpectedly, such topically administered compositions can thus be used to treat pathologies that affect these structures without invasive methods, such as intraocular injection or systemic administration. Examples of pathologies include, but are not limited to, retinopathy including diabetic retinopathy, retinitis pigmentosa, age related macular degeneration, scleritis, uveitis, vasculitis, and oncological diseases affecting the eye such as retinoblastoma, choroidal melanoma, pre-malignant and malignant conjunctival melanoma. Retinoblastoma is a malignant tumor of the retina, typically affecting children under the age of six. Choroidal melanoma is a malignant tumor of the pigmented cells of the choroid. Melanoma of the conjunctiva may be classified as primary acquired melanosis (PAM) with or without atypia, or conjunctival melanoma. For cancers of the eye, treatment with a macrolide antibiotic/mycophenolic acid may provide an anti-angiogenic effect and thereby desirably diminish the blood supply to the tumor. Such treatment may augment or enhance the effects of specific radiation treatments and/or chemotherapeutic agents. For example, macrolide antibiotic and/or mycophenolic acid may be added in polymer form providing extended release to carboplatin, cisplatin, methotrexate, etc., in topical chemotherapy eye drops. Diseases such as diabetic retinopathy, retinitis pigmentosa, and age related macular degeneration are typically chronic so that treatment is prolonged, while diseases such as scleritis, uveitis and vasculitis may be acute with treatment occurring for a shorter duration, that is, over the course of the disease. The invention encompasses both types of treatment, as will subsequently be described.

The topically administered composition must cross ocular structures such as the conjunctiva and sclera to reach structures such as the choroid, retina, and uvea. In transit of the composition, a natural gradient of the active agent(s) may form within the eye. A structure such as the sclera may act as a depot or repository for the active agent(s), providing extended release. Thus, topical administration may provide results similar to a slow release formulation, as will be described. Such formulations desirably decrease the frequency of administration or dosing. For example, patients being treated with the inventive method already have decreased visual acuity, and topical ocular administration of drugs may be difficult and/or uncomfortable for them. Reducing the frequency of administration enhances compliance, while providing a therapeutic dosage of the composition.

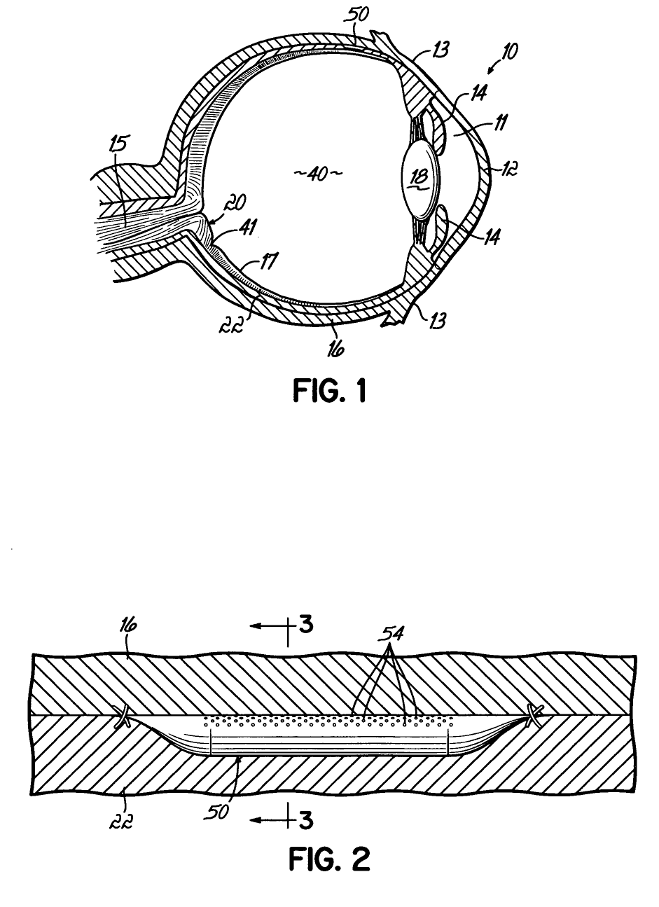

FIG. 1 schematically shows an eye 10 into which the inventive device 50 is placed in approximation to the conjunctiva 13. In other embodiments, the device 50 may be located at any region such that it is fixed to the sclera 16. The locations of the anterior chamber 11, cornea 12, iris 14, optic nerve 15, macula lutea 17, lens 18, retina 20 and choroid 22 are illustrated.

FIGS. 2-10 illustrate various embodiments of the device 50. In any embodiment, the device 50 has lumen 51 for containing one or more agents 52 (e.g., drug) that are released through at least one opening 54. The opening(s) 54 may have a wide variety of sizes and configurations depending on the desires or requirements of a particular application. For example, the opening(s) 54 may be one or more perforations, fenestrations, holes, slits, slots, combinations thereof and other configurations known in the art. The shape of the openings may also vary. For example, the opening(s) 54 may be circular, square, rectangular, elliptical, etc. or combinations thereof in shape. By way of example, FIG. 2 shows a device 50 where opening(s) 54 are configured as circular holes and FIG. 4 shows another embodiment of device 50 where openings 54 are configured as rectangular slots.

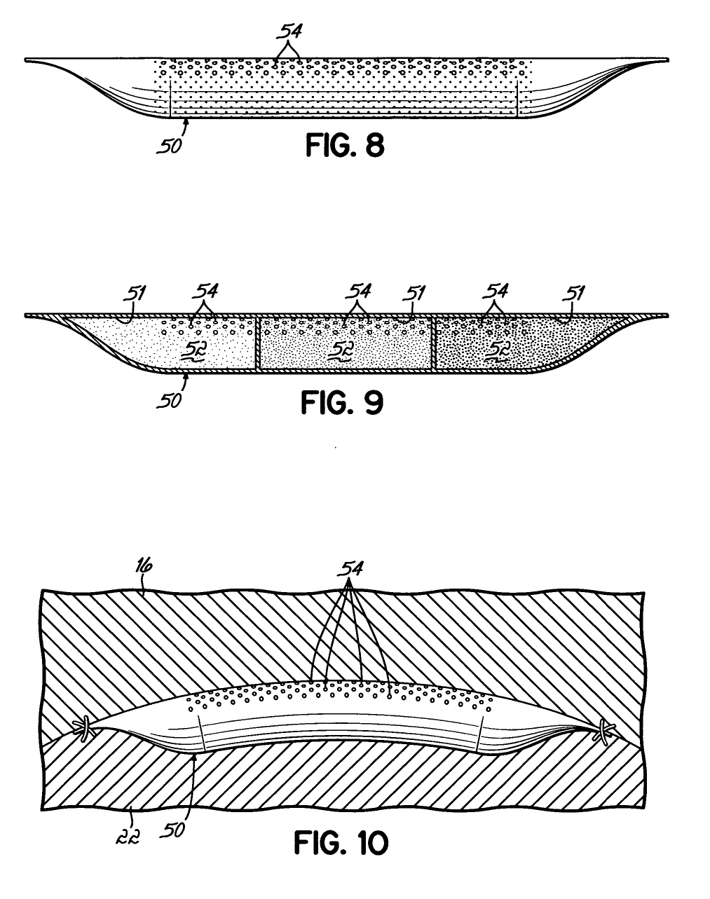

The size of opening(s) 54 may be selected depending on desires or requirements of a particular application. For example, the opening(s) 54 may have an identifiable cross dimension (such as diameter, slot length, etc.) that ranges from less than 1 mm up to several mm (e.g., 5 mm). The size of opening(s) 54 may not only vary from device to device, but may also vary on the same device. Thus, as shown in FIG. 8, some opening(s) 54a on device 50 have a diameter that is larger than other opening(s) 54b on the same device 50. In this way, the small openings 54b may be for water only while the larger openings 54a are for agent release. In one embodiment, the device 50 may have walls or other types of closures that allow selective reduction or prevention of release of the agent 52. To this end, the closures may reduce the size of opening(s) 54 or alternately, completely close opening(s) 54.

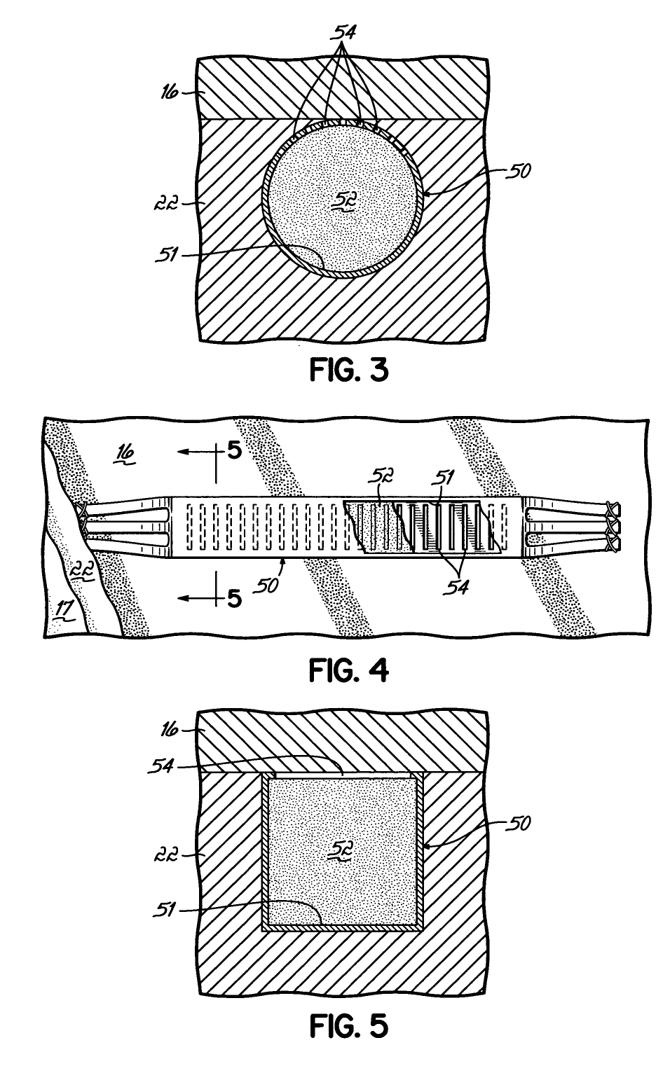

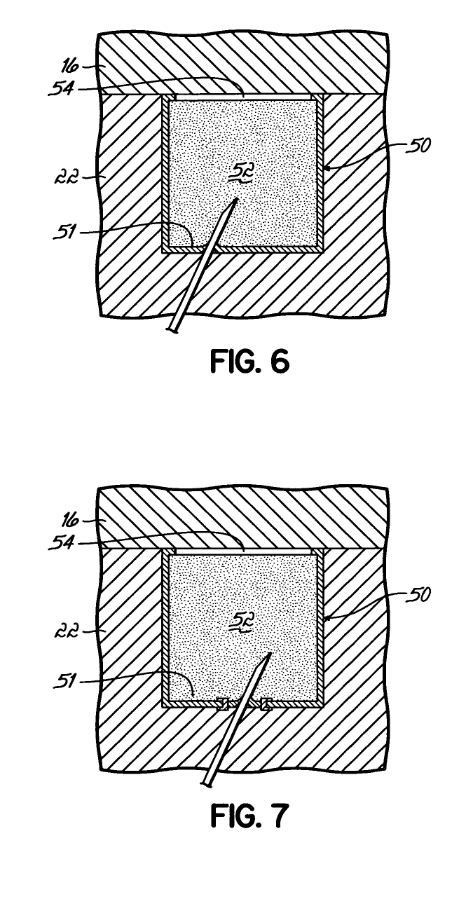

Various configurations (shape, form, material, etc.) of the linear device 50 are also contemplated. For example, device 50 may be a hollow cylinder or tube having a first cross dimension (diameter, width, etc.), ranging from about 1 mm to about 5 mm and a second cross dimension, such as length, from about 2 mm to about 20 mm. By way of example, FIGS. 2 and 3 show a cylindrical device 50 having a first cross dimension shown by diameter 56 and a second cross dimension shown by length 58. In a similar manner, FIGS. 4 and 5 show a square tubular device 50 having a first cross dimension shown by width 60 and a second cross dimension shown by length 62. The two terminal portions, or ends 66, of the device 50 can be tapered, or branched, which facilitates suturing or fixing the device 50 to the sclera 16. For example, FIG. 2 shows a device 50 wherein the terminal ends 66 have a tapered configuration with a suture 68 for securing device 50 to sclera 16. In a similar manner, FIG. 4 shows a device 50 wherein the terminal ends 66 have a branched configuration. FIG. 4 also shows multiple sutures 68 securing device 50 to sclera 16. The device 50 may be made of any biocompatible material; e.g., synthetic, organic, or a mixture. Such materials are known to one skilled in the art. They include, but are not limited to, silicone, a mixture of silicone and other polymers such as a silicone elastomer or a silicone rubber, polymethylmethacrylate, polyolefins such as polypropylene and polyethylene, homopolymers and copolymers of vinyl acetate such as ethylene vinyl acetate copolymer, polyvinylchlorides, homopolymers and copolymers of acrylates such as polyethylmethacrylate, polyurethanes, polyvinylpyrrolidone, 2-pyrrolidone, polyacrylonitriles butadiene, polycarbonates, polyamides, fluoropolymers such as polytetrafluoroethylene and polyvinyl fluoride, polystyrenes, homopolymers and copolymers of styrene acrylonitrile, cellulose acetate, homopolymers and copolymers of acryonitrile butadiene styrene, polymethylpentene, polysulfones, polyesters, polyimides, natural rubber, polyisobutylene, polymethylstyrene, and other non-erodible biocompatible polymers. The device 50 may be hard and rigid, or soft and pliable, or intermediate. In one embodiment, device 50 may be straight, curved, or flexible (as shown in FIGS. 2 and 4) for ease in placing or fitting in the eye 10; e.g., it may be curved so that it fits the curvature of the sclera 16. Such a curved device 50 is shown in FIG. 10. The device 50 may be made of a material that is pliable for insertion and initial fit, but then retains the desired conformation. Such materials are known to one skilled in the art and include, but are not limited to, shape memory alloys and polymers such as nitinol, AB-polymer networks based on oligo(ε-caprolactone) dimethacrylates and n-butyl acrylate. In one embodiment, as shown in FIG. 6, the entire device 50 or at least a wall 70 of the device 50 may be sufficiently flexible to permit resealable penetration of the wall 70 by a needle 72 (e.g. a 27 g or higher needle). This embodiment may, for example, be used to load the device 50 with agent 52, to refill the device 50 with the same agent 52, to add or replace a agent, etc. In an alternate embodiment, the device 50 may contain a port 74 adapted to receive a needle 72. The device 50 may be transparent, translucent, or opaque. If the device 50 is transparent or translucent, its contents, such as agent 52 may be determined by visual inspection to determine if sufficient volume of agent exists for a desired duration of therapy. The device 50 and/or lumen 51 can form a single chamber to hold the agent 52, or it may contain multiple chambers to contain multiple agents 52 in segregated compartments. For example, FIG. 9 shows an embodiment of the device 50 wherein lumen 51 is divided into multiple compartments, three such compartments 76a, 76b and 76c shown in FIG. 9, each having a different agent 52a, 52b and 52c respectively, using dividing walls 78. Those of ordinary skill in the art will recognize that the number of compartments may be varied to suit a particular application. Those of ordinary skill in the art will also recognize that each compartment may have the same or different configurations for opening(s) 54. Any portion or component of device 50 may be on a nanotech scale. These various embodiments are shown in FIGS. 2 through 10.

The agent contained in device 50 is not limited to a macrolide antibiotic and/or mycophenolic acid, although these may be used. In various embodiments, the agent may be one or more of other types of antimicrobial agents (other antibiotics, antifungals, antivirals, etc.), anti-inflammatory agents (e.g., steroids, NSAIDs), anti-proliferative agents (e.g., anti-VEGF), hormones, cytokines, growth factors, antibodies, immune modulators, vectors for gene therapy (e.g., viral or plasmid vectors), oligos (e.g., RNA duplexes, DNA duplexes, RNAi, aptamers, immunostimulatory or immunoinhibitory oligos, etc.), enzymes, enzyme inhibitors, immune modulators, etc. The agent may be in a liquid or semi-liquid form, a suspension, an emulsion, etc. Any of the above agents 52 may be formulated as nanoparticles or nanocrystals of pharmaceutically active compounds, and/or nanoscale dispersions, encapsulations, and emulsions (e.g., to limit or prevent aggregation of reaggregation or crystals, to incorporate a stabilizer, etc.). The agents 52 may be combined with albumin or another non-toxic solvent to form nanoparticles in a solvent-free formulation of a toxic drug. The agents 52 may be formulated as sugar-derived nanocompounds that may shield proteins and small molecules from rapid breakdown. The drugs may be rendered more soluble in a nanocrystal formulation by decreasing drug particle size and hence increasing the surface area thereby leading to an increase in dissolution. These techniques are known to one skilled in the art as disclosed in, for example, U.S. Pat. Nos. 6,822,086; 6,753,006; 6,749,868; 6,592,903; 6,537,579; 6,528,067; 6,506,405; 6,375,986; 6,096,331; 5,916,596; 5,863,990; 5,811,510; 5,665,382; 5,560,933; 5,498,421; 5,439,686; and 5,362,478; and U.S. patent application Ser. Nos. 10/106,117; 60/147,919; and 08/421,766, each of which is expressly incorporated by reference herein in its entirety.

The device 50 may indicate, or may contain an indicator for, the amount or volume of agent 50 remaining in the device 50. For example, an indication may be of the need to refill the device 50, or signal that release of agent 52 occurred, or signal that the device 50 is empty, etc. The indictor may be, e.g., a chromogen.

In one embodiment, the device 50 may be fabricated to be externally regulated. For example, dosing may be controlled by a software program that communicates with a microchip associated with the device 50. The program may be accessed, verified, altered, monitored, etc., even from a remote location.

In embodiments, agent release from the device 50 may be pre-set, or may be manually regulated at the point of use, or may regulated from a remote location. This may include volume, duration, rate, release intervals, etc. In one embodiment, agent release is remotely controlled by electric stimulation. For example, the opening may be partially or completely associated with a piezoelectric film, an electric erosion barrier, etc. Upon electric stimulation, the film or barrier is disrupted sufficiently to allow at least a portion of the contents of the device 50 to egress through the opening(s) 54. If more than one opening 54 is present, each opening 54 may be associated with a film, barrier, etc. that requires different stimulation levels to disrupt, allowing selective control of agent delivery. The film or barrier may cover all or part of the opening 54, or be located adjacent an opening 54, in its association with the device 50. In another embodiment, the device 50 is remotely controlled by microactivation, whereby the patient is fitted with a receiving device such as an antenna, and a radiofrequency identification (RF-ID) chip carrying a microactivator for causing agent release. An RF-ID interrogator is used to interrogate the receiving device, for example, from a remote location, providing power to the RF-ID chip and causing the RF-ID chip to trigger the microactivator by delivering an appropriate coded instruction to the RF-ID chip via radiofrequency signals.

Radio frequency (RF) telemetry may be used to remotely activate the device to release agent, as known to one skilled in the art. The circuitry, programming, and other components and their implementation are described in, e.g. U.S. Pat. No. 5,170,801 where a circuit in a capsule device receives RF signals and causes drug release from openings in the device; U.S. Pat. No. 5,820,589 where RF telemetry is used to program and/or reprogram power and/or flow rate information to an implanted pump to release a drug, with the pump containing an antenna and circuitry to receive a signal transmitted by an external remote device placed over the skin of the patient; upon receiving a signal, the circuitry changes the operating parameters and the new settings remain in place until new programming instructions are received by RF signals or other non-invasive telemetry in the circuitry; U.S. Pat. No. 5,312,453 describing an external programmer device that transmits RF encoded signals to an implanted device using programming that allows remote selection of parameters and settings for the implanted device; and U.S. Pat. No. 6,824,561, disclosing a hand-held device using RF, infrared, acoustic pulsed, or magnetic activating means where a surgeon, physician, or patient holds the device over the implant site and activates the device to release agent(s). Each of these patents is expressly incorporated by reference herein in its entirety.

The embodiments described, as well as others, can be adapted by one skilled in the art. As described, the remote activating device may contain a microprocessor and at least one antenna to transmit RF signals to the implanted device. A programming circuit in the implanted device may contain at least one antenna to receive transmitted signals from the remote device and, upon detection of a signal, the programming circuit may cause release of agent from an opening in the implanted device. As a result, a, physician is able to remotely activate the implanted device to release the agent. Additional safety precautions may also be incorporated by one skilled in the art. As one example, the programming circuitry may be configured to respond only to a specific RF signal in order to avoid accidental activation of the implanted device. As another example, the programming circuitry may be configured to incorporate pre-determined dosage information into the remote device in order to prevent remote activation of the implanted device after a maximum dosage has been already released.

RF signals or other telemetry may also serve as a power supply for the implanted device, circuit, and/or any other components. Thus, while operating the remote device, power may be transmitted to the implanted device via the transmitted RF signal, and release of agent may cease when the individual operating the remote device causes it to stop transmitting a signal (i.e., removing the power supply). Various modifications may be made to the embodiments above as known to one skilled in the art.

In one embodiment, the device 50 may be formulated to release the agent 52 by electromotive drug administration, also referred to as iontophoresis, using a small electrical current passed through the eye 10. In this embodiment, the inventive device 50 contains an electrode, i.e., an anode and/or cathode depending upon the charge state of the agent(s). The device 50 may contain both anode and cathode to accommodate different agents or drugs contained in different compartments 76 of the device 50. An electrode of opposite polarity (cathode and/or anode) is inserted on the sclera 16 at a site opposite device 50. The flow of current is regulated externally to the eye 10 by an energy source. When current is applied, an electrical potential difference is generated between the two electrodes, providing agent to the eye 10. Such administration may permit a relatively high concentration of agent to be delivered diffusively to the affected tissue, rather than being localized at the site of administration. The dose of agent delivered depends upon the current and duration selected. In one embodiment, a current between about 0.5 mA and about 4 mA is applied for between a few seconds to about 20 min.

Iontophoresis delivery itself has no side effects and there is no pain associated with agent administration. Thus, it may be used in any embodiment of the device 50, including those in which the device 50 is externally regulated, and in embodiments where a supratherapeutic concentration of agent 52 is to be delivered.

For implanting and fixing the device 50, an anesthetic is administered to the patient (e.g., topical, local, etc.) as known to one skilled in the art. A relatively small (about 5 mm) incision is made in the peribulbar conjunctiva 13 such that a pocket is created between the conjunctiva 13 and sclera 16. In one embodiment, a device 50 is implanted in the pocket and fixed (e.g., by one or more sutures 68, using a biocompatible sealant, adhesive, etc.) to the scleral wall. In another embodiment, a tunnel is created in the sclera 16 and the device 50 is positioned within the tunnel. For example, a cylindrical or tube-shaped device 50 within such a tunnel does not require suturing. In one embodiment, the device wall may be colored so that it is visible. In one embodiment, a pre-filled device 50 is inserted. In a multichamber device 50, one or more of the chambers 76 may be filled prior to insertion. In one embodiment, the device 50 is inserted and is thereafter filled with agent 52.

In one embodiment, a concentration of macrolide antibiotic and/or mycophenolic acid in a pharmaceutically acceptable topically administered solution may range from about 0.5%^{w/v} to about 10%^{w/v}. In another embodiment, a concentration of macrolide antibiotic and/or mycophenolic acid in a pharmaceutically acceptable topically administered solution may range from about 3%^{w/v} to about 5%^{w/v}. In another embodiment, a concentration of macrolide antibiotic and/or mycophenolic acid in a pharmaceutically acceptable topically administered solution may range from about 1%^{w/v} to about 3%^{w/v}. In another embodiment, a concentration of macrolide antibiotic and/or mycophenolic acid in a pharmaceutically acceptable topically administered solution may range from about 3%^{w/v} to about 10%^{w/v}. In any of the above embodiments, the patient is typically instructed to periodically administer the solution, from once per day up to several times per day, over the course of the disease. In one embodiment, the composition may be administered daily at bedtime. It will be appreciated that some patients with a chronic disease will require continued treatment over many years.

The inventive composition may be used in physiologic ophthalmic irrigating solutions. One example is Balanced Salt Solution (BSS®, available from Alcon Laboratories, Randburg, South Africa), containing per ml 0.64% sodium chloride, 0.075% potassium chloride, 0.048% calcium chloride, 0.03% magnesium chloride, 0.39% sodium acetate, and 0.17% sodium citrate dihydrate, as well as sodium hydroxide and/or hydrochloric acid to adjust pH, and water for injection. Another example is Ocular Irrigation Solution® (Allergan, Irvine Calif.). Another example is lactated Ringer's solution. Another example is a normal saline solution. Another example is normal saline adjusted to pH 7.4 with sodium bicarbonate.

The inventive composition may also be used in ophthalmic volume replacement solutions. For example, it may be introduced into the posterior chamber of the eye to replace the vitreous that is removed during the repair of retinal disorders (vitrectomy).

The inventive composition may also be introduced into the lens capsule during cataract surgery. A cloudy and discolored lens, referred to as a cataract, causes decreased vision and treatment requires that the lens be surgically removed. Cataract surgery usually involves phacoemulsification of the diseased lens inside the capsule, aspiration of the emulsified material, irrigation, and insertion of a replacement intraocular lens (IOL) within the capsule.

Following cataract surgery, there is frequently opacification of the posterior capsule which also diminishes visual acuity. Surgical techniques to minimize posterior capsule opacification have variable success, and patients undergoing cataract surgery may require an additional procedure to attend to the capsular opacification that subsequently occurs.

A complication for IOL implantation is post-operative opacification. This occurs as a result of lens epithelial cells (LEC) which migrate around the posterior capsule, and may be due to lack of maximum contact between the IOL optic and the posterior capsule. In children treated for pediatric cataracts, leaving the posterior capsule intact after IOL implantation predisposes them to secondary cataract formation and severe visual axis opacification (VAO). This usually requires surgery to prevent VAO and an anterior vitrectomy to maintain a clear visual axis during pediatric IOL surgery. Thus, reduction in the extent of cell migration and/or cell proliferation following cataract surgery is desirable.

In this embodiment of the invention, an irrigating or volume replacement solution containing at least one macrolide antibiotic and/or mycophenolic acid is administered to the capsule with or before inserting the replacement lens. Without being bound by any theory, the macrolide antibiotic and/or mycophenolic acid may reduce posterior capsular opacification and visual axis opacification by its inhibitory effect on ocular cell proliferation and cell migration.

The macrolide antibiotic and/or mycophenolic acid can also be provided on a device, such as a contact lens applied to the exterior surface of an eye, or a lens that will be implanted within a patient's eye. Implantable lenses include any IOL used to replace a patient's diseased lens following cataract surgery, including but not limited to those manufactured by Bausch and Lomb (Rochester N.Y.), Alcon (Fort Worth Tex.), Allergan (Irvine Calif.), and Advanced Medical Optics (Santa Ana Calif.). The system provides a therapeutic replacement lens ready for surgical implantation in a patient. When the lens is implanted within the lens capsule, the antibiotic and/or mycophenolic acid provides therapeutic effects (e.g., anti-cell proliferative effects, anti-inflammatory effects, etc) to the eye.

A concentration of the macrolide antibiotic and/or mycophenolic acid within the capsule is provided to achieve the previously described therapeutic effect. In one embodiment, the concentration ranges from about 20 μg/ml (about 0.002%^{w/v}) to about 2000 μg/ml (about 0.2%^{w/v}). In another embodiment, the concentration ranges from about 200 μg/ml (about 0.02%^{w/v}) to about 2000 μg/ml (about 0.2%^{w/v}). In another embodiment, the concentration ranges from about 20 μg/ml (about 0.002%^{w/v}) to about 200 μg/ml (about 0.02%^{w/v}).

In another embodiment, the IOL or device contains a concentration of the macrolide antibiotic and/or mycophenolic acid up to about 2%^{w/v} formulated so that the concentration in the eye at any time does not exceed about 40 μg/ml. For example, the intraocular concentration of the active agent(s) at any time may be in the range of about 10 μg/ml to about 30 μg/ml. Such formulation methods are known to one skilled in the art and include, but are not limited to, extended release formulations subsequently described.

The contact lens or IOL may be made of hydrophobic or hydrophilic material. The type of material determines whether the lens cannot fold, is rigid and requires a large incision to insert, or is flexible to allow the lens to be rolled, compressed, or folded for insertion through a smaller incision. The most common materials used in lenses are various chemical modifications of silicon, hydrophobic acrylates, hydrophobic acrylates, and hydrogels which contain water to impart gel-like characteristic to the material. Each of these can be formulated or treated to contain a solution containing a macrolide antibiotic and/or mycophenolic acid.

In one embodiment, the contact lens or implantable IOL is packaged in an ophthalmically acceptable medium which contains the macrolide antibiotic and/or mycophenolic acid. For example, a porous hydrogel lens (e.g., Hydroview®, Bausch & Lomb Surgical, Rochester N.Y.) retains the macrolide antibiotic and/or mycophenolic acid within the pores. Upon application of the contact lens or insertion/implantation of the lens into the lens capsule, the macrolide antibiotic and/or mycophenolic acid is released. An ocular device containing agent(s) in a slow-release system provides extended therapy, for example, over a post-surgical recovery period as the actives are slowly released through the porous elements. It may also be the administration method of choice in some patients, such as patients who are elderly, who cannot reliably self-administer topical ocular medications, who must receive chronic therapy, etc.

In another embodiment, a contact lens or implantable lens is coated to provide the macrolide antibiotic and/or mycophenolic acid. This embodiment may be used with a non-hydrogel hydrophilic lens, a hydrophobic lens, a lens made from an acrylic material (e.g., AcrySof®); Alcon, Fort Worth Tex.; Sensar®; Advanced Medical Optics, Santa Ana Calif.), a silicone lens (e.g., CeeOn®, Pharmacia & Upjohn Company, Pickering Ohio), etc. Coating and/or incorporation procedures that may be used are known to one skilled in the art; for example, as disclosed in U.S. Pat. Nos. 6,238,799; 6,179,817; 6,306,422; and 6,258,856, each of which is incorporated by reference herein in its entirety. The macrolide antibiotic and/or mycophenolic acid may be added to the storage solution during packaging of the lens, or may be incorporated into the manufacture of the lens. For example, the macrolide antibiotic and/or mycophenolic acid may be incorporated into either or both the hydration fluid in the formation of a hydrophilic or hydrogel lens, or in the storage solution. In another example, the macrolide antibiotic and/or mycophenolic acid may be in an acceptable encapsulated form in a hydrogel IOL for extended long term release.

The inventive composition may also be used as an ocular wash solution, for example, to clear the surgical field during intraocular surgery.

In each of the above embodiments, any macrolide antibiotic alone or in combination may be used. Embodiments of the invention include various ocular-compatible concentrations of the macrolide antibiotic(s) and/or mycophenolic acid sufficient to provide an anti-inflammatory, anti-proliferative, anti-cell migration, anti-fungal, etc. effect. Concentrations may depend upon the use for the composition, as is known to one skilled in the art. Thus, in these embodiments, the invention is not limited to a specific concentration of macrolide antibiotic and/or mycophenolic acid. In general, the macrolide antibiotic and/or mycophenolic acid is present in the ocular solution at concentrations ranging from about 1 ng/ml (about 0.0000001%^{w/v}) to about 200 μg/ml (about 0.02%^{w/v}). In one embodiment, the macrolide antibiotic and/or mycophenolic acid is present in the ocular solution at a concentration of about 1 μg/ml (about 0.0001%^{w/v}). For use to reduce capsular opacification following cataract surgery, concentrations ranging from about 1 μg/ml (about 0.0001%^{w/v}) to about 200 μg/ml (about 0.02%^{w/v}), or from about 20 μg/ml (about 0.002%^{w/v}) to about 200 μg/ml (about 0.02%^{w/v}), may be used. The properties of the macrolide—and/or mycophenolic acid-containing ocular solution are compatible with ocular tissues.

The macrolide antibiotic and/or mycophenolic acid may be formulated with a viscoelastic substance such as hyaluranic acid, or may be contained in microspheres, macrospheres, microvesicles, macrovesicles, microcapsules, macrocapsules, liposomes, etc., as described in co-pending U.S. patent application Ser. No. 10/631,143 which is expressly incorporated by reference herein in its entirety. This embodiment may be used with solutions administered to prevent capsular opacification following cataract surgery, as previously described.

Liposomes may be prepared from dipalmitoyl phosphatidylcholine (DPPC), for example, from egg phosphatidylcholine (PC), a lipid with a low heat of transition. Liposomes are made using standard procedures as known to one skilled in the art. The macrolide antibiotic(s), in amounts ranging from nanogram to microgram quantities, or higher, is added to a solution of egg PC, and the lipophilic drug binds to the liposome.

A time-release drug delivery system may be administered intraocularly to result in sustained release of the macrolide antibiotic(s) over a period of time. The formulation may be in the form of a vehicle, such as a micro- or macro-capsule or matrix of biocompatible polymers such as polycaprolactone, polyglycolic acid, polylactic acid, polyanhydrides, polylactide-co-glycolides, polyamino acids, polyethylene oxide, acrylic terminated polyethylene oxide, polyamides, polyethylenes, polyacrylonitriles, polyphosphazenes, poly(ortho esters), sucrose acetate isobutyrate (SAIB), and other polymers such as those disclosed in U.S. Pat. Nos. 6,667,371; 6,613,355; 6,596,296; 6,413,536; 5,968,543; 4,079,038; 4,093,709; 4,131,648; 4,138,344; 4,180,646; 4,304,767; 4,946,931, each of which is expressly incorporated by reference herein in its entirety, or lipids that may be formulated as microspheres or liposomes. A microscopic or macroscopic formulation may be administered through a needle, or may be implanted by suturing within the eye, for example, within the lens capsule. As an illustrative example, sirolimus may be mixed with polyvinyl alcohol (PVA), the mixture then dried and coated with ethylene vinyl acetate, then cooled again with PVA. In a formulation for intraocular administration, the liposome capsule degrades due to cellular digestion providing a slow release drug delivery system, allowing the patient a constant exposure to the drug over time.

A time-release microscopic or macroscopic formulation may also be topically administered. The sustained-release antibiotic(s) and/or mycophenolic acid accumulates at concentrations in ocular structures such as the choroid or retina sufficient to effect treatment and diseases affecting these structures.

Delayed or extended release properties may be provided through various formulations of the vehicle (coated or uncoated microsphere, coated or uncoated capsule, lipid or polymer components, unilamellar or multilamellar structure, and combinations of the above, etc.). Other variables may include the patient's pharmacokinetic-pharmacodynamic parameters (e.g., body mass, gender, plasma clearance rate, hepatic function, etc.). The formulation and loading of microspheres, microcapsules, liposomes, etc. and their ocular implantation are standard techniques known by one skilled in the art, for example, the use a ganciclovir sustained-release implant to treat cytomegalovirus retinitis, disclosed in Vitreoretinal Surgical Techniques, Peyman et al., Eds. (Martin Dunitz, London 2001, chapter 45); Handbook of Pharmaceutical Controlled Release Technology, Wise, Ed. (Marcel Dekker, New York 2000), the relevant sections of which are incorporated by reference herein in their entirety.

Examples of macrolide antibiotics that may be used for intraocular administration include, but are not limited to, tacrolimus, Cyclosporin A, sirolimus, ascomycin, and everolimus. Tacrolimus (Prograf®, Fujisawa Healthcare, Deerfield, IL; FK-506), a macrolide immunosuppressant produced by Streptomyces tsukubaensis, is a tricyclo hydrophobic compound that is practically insoluble in water, but is freely soluble in ethanol and is very soluble in methanol and chloroform. It is available under prescription as either capsules for oral administration or as a sterile solution for intravenous administration. The solution contains the equivalent of 5 mg anhydrous tacrolimus in 1 ml of polyoxyl 60 hydrogenated castor oil (HCO-60), 200 mg, and dehydrated alcohol (USP, 80.0%^{v/v}), and must be diluted with a solution of 0.9% NaCl or 5% dextrose before use.

Tacrolimus has been used for topical administration to treat a variety of dermatoses. Topical administration of tacrolimus at doses ranging from 0.03%-0.3% resulted in significant clinical improvement in atopic dermatitis after 2-3 weeks treatment, and tacrolimus treatment of other dermatologic diseases shows promise. Tacrolimus, like cyclosporine, blocks the signal transduction pathway needed to induce interleukin-2 gene expression and thereby activate T lymphocytes. In addition to suppressing T cell activation, tacrolimus inhibits anti-IgE-triggered histamine release and inhibits prostaglandin D2 synthesis in human skin mast cells. While oral administration produces limiting adverse effects (systemic immunosuppression, infection, neural toxicity, nephrotoxicity, and hypertension), topical administration for treatment of dermatoses at concentrations up to 0.3% showed no significant difference in effects between treated and control groups. In addition, tacrolimus is well tolerated locally and only occasionally causes mild irritation.

The use of tacrolimus as a specific medicament for treatment of ocular disease has been disclosed in U.S. Pat. No. 6,489,335 and co-pending U.S. patent application Ser. No. 10/247,220, each of which is expressly incorporated by reference herein in its entirety. For example, tacrolimus may be contained in an aqueous-based cream excipient for topical application, or it may be injected intraocularly, or it may be administered surgically as an ocular implant.

None of these publications disclose the topical ocular administration of supratherapeutic concentrations of a macrolide antibiotic and/or mycophenolic acid, either alone or with other agents such as chemotherapeutic agents and/or inhibitors of cyclooxygenase, at the disclosed doses and formulations for treating ocular pathologies such as diabetic retinopathy, retinitis pigmentosa, age related macular degeneration, uveitis, vasculitis, retinoblastoma, choroidal melanoma, pre-malignant and malignant melanoma of the conjunctiva, as in the inventive method.

Cyclosporin A (cyclosporine, topical formulation Arrestase®, Allergan Inc.) is a cyclic peptide produced by Trichoderma polysporum. It is available commercially, for example, from Sigma-Aldrich (St. Louis Mo.). It is an immunosuppressant and acts in a particular subset of T lymphocytes, the helper T cells. Cyclosporin A exerts an immunosuppressant effect by inhibiting production of the cytokine interleukin 2. Each of Cyclosporin A and tacrolimus, another immunosuppressant, produce significant renal and hepatic toxicity when each is administered systemically; because of this toxicity, they are not administered together.

Cyclosporin A has been administered to treat ocular conditions such as glaucoma, corticosteroid-induced ocular hypertension, allograft rejection, infections, and ocular surface disease. Its use has been reported for the treatment of uveitis (inflammation of the uvea) by topical, intravitreal or systemic administration with doses of 0.05%, 0.1%, and 0.5%. Cyclosporin A has good penetration into the cornea but not into the anterior chamber, and does not increase intraocular pressure or cause cataracts. Its known toxicity had previously limited its use for other ocular diseases.

The use of Cyclosporin A as a specific medicament for treatment of ocular disease with reduced toxicity has been described in co-pending U.S. patent application Ser. No. 10/289,772, which is expressly incorporated by reference herein in its entirety.

Sirolimus, also known as rapamycin, RAPA, and Rapamune®, is a triene macrolide antibiotic derived from Streptomyces hydroscopicus and originally developed as an antifungal agent. Subsequently, it has shown anti-inflammatory, anti-tumor, and immunosuppressive properties. Ascomycin, also known as pimecrolimus, Immunomycin, and FR-900520, is an ethyl analog of tacrolimus and has strong immunosuppressant properties. It inhibits Th1 and Th2 cytokines, and preferentially inhibits activation of mast cells, and is used to treat contact dermatitis and other dermatological conditions. Sirolimus and ascomycin are commercially available, e.g., A.G. Scientific, Inc. (San Diego, Calif.).

Regarding its immunosuppressive potential, sirolimus has some synergetic effect with Cyclosporin A. It has been reported that sirolimus has a different mode of action compared to Cyclosporin A and tacrolimus. All three agents are immunosuppresants which affect the action of immune cell modulators (cytokines), but do not affect the immune cells themselves. However, while all three agents affect immune cell modulators, they do so differently: Cyclosporin A and tacrolimus prevent synthesis of cytokine messengers, specifically interleukin-2, while sirolimus acts on cytokine that has already been synthesized, preventing it from reaching immune cells.

Sirolimus inhibits inflammation by acting on both T-lymphocytes and dendritic cells. The latter are the first cells to recognize antigens. Sirolimus blocks the growth of dendritic cells and a number of other cells, such as tumors and endothelial cells, which are activated by the tumor cell releasing vascular endothelial growth factor (VEGF). VEGF is a central regulator of angiogenesis (formation of new blood vessels from pre-existing vessels) and vasculogenesis (development of embryonic vasculature through an influence on endothelial cell differentiation and organization). Diseases that are characterized by abnormal angiogenesis and vasculogenesis, such as some cancers and some ocular diseases, may show abnormal production of VEGF. Thus, control of VEGF function may be one means to control or treat these diseases. Sirolimus has also been used in the prevention of smooth muscle hyperplasia after coronary stent surgery. The use of sirolimus and ascomycin as specific medicaments for treatment of ocular disease has been disclosed in co-pending U.S. patent application Ser. No. 10/631,143, which is expressly incorporated by reference herein in its entirety.

Everolimus, also known as RAD-001, SCZ RAD, Certican™ (Novartis, Basel Switzerland), is an analog of sirolimus but is a new and distinct chemical entity. It is an oral immunosuppressant that inhibits growth factor-induced cell proliferation and thus reduces acute organ rejection and vasculopathy, the proliferation of smooth muscle cells in the innermost wall of grafts that restricts blood supply.

Mycophenolic acid (MPA) is the active compound formed following the administration of mycophenolate mofetil (MMF). The prodrug is the morpholinoethyl ester of mycophenolic acid. Mycophenolic acid is an antileukemic and immunosuppressant agent used in patients undergoing chemotherapy for cancer and in transplant recipients.

The topical ocular administration of these agents, either alone, in combination, or with chemotherapeutic agents or cyclooxygenase inhibitors, at the disclosed concentrations and formulations to treat ocular pathologies such as diabetic retinopathy, retinitis pigmentosa, age related macular degeneration, uveitis, vasculitis, retinoblastoma, choroidal melanoma, pre-malignant and malignant conjunctival melanoma has not been reported.

The addition of these agents, either alone or in combination, to invasively administered ocular solutions according to the invention provides beneficial anti-inflammatory, anti-proliferative, anti-cell migration, anti-angiogenic, antimicrobial, and antifungal properties.

It will be appreciated that the invention encompasses the use of macrolide antibiotics and/or mycophenolic acid, in addition to those previously described, in an ocular solution. These include, for example, the known antibiotics erythromycin and its derivatives such as azithromycin and clarithromycin, lincomycin, dirithromycin, josamycin, spiramycin, diacetyl-midecamycin, troleandomycin, tylosin, and roxithromycin. The invention also includes new macrolide antibiotic scaffolds and derivatives in development, including but not limited to the ketolides ABT-773 and telithromycin as described by Schonfeld and Kirst (Eds.) in Macrolide Antibiotics, Birkhauser, Basel Switzerland (2002); macrolides derived from leucomycins, as described in U.S. Pat. Nos. 6,436,906; 6,440,942; and 6,462,026 assigned to Enanta Pharmaceuticals (Watertown Mass.); and lincosamides.

In addition to the above described uses, the invention comprises ocular solutions for topical (non-invasive) ocular administration with everolimus, erythromycin, azithromycin, clarithromycin, lincomycin, dirithromycin, josamycin, spiramycin, diacetyl-midecamycin, tylosin, roxithromycin, and mycophenolic acid, as well as the previously described new macrolide antibiotic scaffolds and derivatives in development, including but not limited to the ketolides ABT-773 and telithromycin, macrolides derived from leucomycins, and lincosamides.

The macrolide antibiotics are included with ocular solutions for any use. They may be included with topical ocular solutions containing chemotherapeutic agents for treating ocular malignancies or pre-malignant conditions. They may be included with topical ocular solutions containing inhibitors of cyclooxygenase for reducing ocular inflammation. In one embodiment, age related macular degeneration is treated by administering a topical ocular formulation containing at least one macrolide antibiotic and/or mycophenolic acid and at least on cyclooxygenase inhibitor. The cyclooxygenase inhibitor(s) may be present in a concentration of 0.5% to 20% of the composition. Inhibitors of cyclooxygenase (COX inhibitors) are well known (e.g., Viiox®, Celebrex®) and include, but are not limited to, ibuprofen, indomethacin, piroxicam, and tranylcypromine HCl. The macrolide antibiotic may be added together or separately as individual components in the preparation of an ocular solution. Alternatively, a solution of the macrolide antibiotic may be prepared and then added to the ocular solution. The solutions may be commercial irrigating solutions that contain other known components, such as various anions and cations, buffers to regulate pH, adenosine, calcium, glucose, bicarbonate, dextrose, dextran 40 (a low molecular weight colloidal osmotic agent), gentamicin, dexamethasone, selenium, zinc, and gluconide. The macrolide antibiotic may be added to commercial ocular lubricating solutions, such as artificial tears. The macrolide antibiotic may be included with commercial ocular wash solutions. The macrolide antibiotic may be included with contact lens wash, rinse, and wetting solutions. Any solution for ocular administration, either administration to the exterior surface of the eye or to one of the interior chambers of the eye, may contain the macrolide antibiotic.

The invention is also not limited to human use, and encompasses the use of ocular solutions containing at least one macrolide antibiotic for veterinary use. For example, lincosamides have been used in animals; an ocular solution containing a lincosamide may be used as a veterinary irrigation solution, volume replacement solution, topical wash or lubricant solution, etc.

The invention provides general purpose ocular solutions in the form of eye drops, eye washes, eye irrigating solutions, volume replacement solutions, contact lens solutions, etc. that contain one or more of the above macrolide antibiotics. In various embodiments, the ocular solution may be in single or multi-dose containers (e.g., 10 ml, 20 ml, 30 ml, 500 ml).

Other variations or embodiments of the invention will also be apparent to one of ordinary skill in the art from the above figures and descriptions. Thus, the forgoing embodiments are not to be construed as limiting the scope of this invention.

1. A method for ocular drug delivery comprising providing to an eye of a patient a delivery device having at least one opening for release of an agent contained within at least one lumen of the device, and fixing the device to the sclera such that the agent is released into the sclera through the opening.

2. The method of claim 1 wherein the device is fixed to the sclera substantially adjacent the conjunctiva or under the conjunctiva.

3. The method of claim 1 wherein release of agent from the device is regulated remotely.

4. The method of claim 1 wherein an electrically-disruptable material is associated with the opening and an electrical stimulus disrupts the material to release the agent

5. The method of claim 1 wherein the device further comprises at least one electrode and an opposite electrode is fixed to the sclera substantially opposite the device, and a current is generated to provide release of the agent from the device by iontophoresis.

6. The method of claim 1 wherein the agent is selected from the group consisting of an antibiotic, anti-inflammatory, anti-proliferative, hormone, cytokine, growth factor, antibody, immune modulator, vector for gene therapy, oligo, enzyme, enzyme inhibitors, and combinations thereof.

7. The method of claim 1 wherein the lumen is separated into at least a first compartment and a second compartment.

8. The method of claim 7 wherein a drug is contained in each compartment and release of the drug from each compartment is independently controlled.

9. The method of claim 1 wherein the contents of the lumen are detectable in the affixed device.

10. The method of claim 1 wherein the device is affixed to an outer scleral wall.

11. The method of claim 1 wherein the device is affixed by an adhesive, a sealant, or combinations thereof.

12. The method of claim 1 wherein the device is inserted into a tunnel created in the sclera.

13. The method of claim 1 wherein a pre-filled device is inserted.

14. The method of claim 1 wherein the device further comprises a port and the device is refillable through the port.

15. The method of claim 1 wherein the device is encapsulated.

16. The method of claim 1 wherein at least a portion of the device or the agent is in a nanotechnology formulation.

17. An ocular drug delivery device comprising at least one lumen for containing a pharmaceutically active agent, at least one controllable opening, a subtantially linear body, the device shaped for affixing to a sclera in approximation to a conjunctiva or for locating within a tunnel created in a sclera, the device capable of controllably releasing agent contained in the lumen from the opening.

18. The device of claim 17 having at least two lumens.

19. The device of claim 17 further comprising means associated with the opening for control of drug release from a remote location.

20. The device of claim 17 at least a portion of which is nanotechnology.
